(19) United States
(12) Patent Application Publication
(10) Pub. No.: US 2012/0065140 A1
(43) Pub. Date: Mar. 15, 2012

(54) Biomarkers for the Diagnosis of Autoimmune Disease

(76) Inventors: Jörg J. Goronzy, Palo Alto, CA (US); Cornelia M. Weyand, Stanford, CA (US)

(21) Appl. No.: 13/231,282

(22) Filed: Sep. 13, 2011

Related U.S. Application Data
(60) Provisional application No. 61/403,347, filed on Sep. 13, 2010.

(51) Int. Cl. A61K 38/02; A61P 19/02; G01N 21/76; C40B 30/00; C12Q 1/48; G01N 33/573

(52) U.S. Cl. 514/16.6; 435/15; 435/7.24; 506/7

(57) ABSTRACT

Compositions and methods are provided for diagnosis and treatment of rheumatoid arthritis. Defects in T cell receptor signaling lower the activation threshold of RA T cells, thus predisposing for a failure in maintenance of immune tolerance. Overexpression of B-Raf and/or K-Ras in CD4 T cells lowers the threshold to respond to TCR triggering in the absence of costimulation and increases responses to citrullinated peptides and other autoantigens.

### GOVERNMENT RIGHTS

This invention was made with Government support under grant nos. AR41974, AR42527 and AI44142 awarded by the National Institutes of Health. The Government has certain rights in this invention.

### BACKGROUND OF THE INVENTION

Autoimmune disease occurs when a specific adaptive immune response is mounted against self antigens. The consequence is that the effector pathways of immunity cause chronic inflammatory injury to tissues, which may prove lethal. Autoimmunity may be initiated by the activation of antigen-specific T cells, although the specific triggering mechanism remains unknown.

Although the etiology of most autoimmune diseases is unknown, it has become apparent that interaction and communication between regulatory and effector cells of the immune system is important in triggering and maintaining autoimmune inflammation, as well as potentially causing tissue damage tissue damage. As key elements of this communication network, cytokines and chemokines orchestrate the recruitment, survival, expansion, effector function and contraction of autoreactive lymphocytes in autoimmunity.

For example, the most visible target of the inflammatory process in rheumatoid arthritis is the synovial membrane; joint pain and swelling and eventually, irreversible damage to cartilage, tendons, and bones dominate the clinical presentation. Disease-related pathologies are, however, not limited to synoviocytes, tissue-infiltrating immune cells and other cell constituents of the arthritic joint but involve the global immune system. The inflammatory process characteristic of RA affects non-articular tissues causing a wide spectrum of disease. The systemic nature of the disease is reflected in deregulated immune responses.

For decades, rheumatoid factors, autoantibodies to IgG Fc have been used as diagnostic tools for RA. More recently, an immune response to citrullinated self-peptides was found to be characteristic for RA; anti-citrullinated antibodies have equal sensitivity and higher specificity than RF in the diagnosis of RA. While the roles of these autoimmune responses in the disease process are undetermined, the nature of the autoantigens suggests that RA patients have a peripheral tolerance defect, in particular to neoantigens created by posttranslational protein modifications that accumulate throughout life (see Goronzy and Weyand (2009) Arthritis Res Ther 11:249). Such a generalized defect may also explain the therapeutic benefit of CTLA4-Ig treatment (see Weyand and Goronzy (2006) Nat Clin Pract Rheumatol 2:201-210). CTLA4-Ig blocks the CD28-CD80/86 receptor-ligand interaction and therefore inhibits costimulatory signals that are particularly important for primary T cell responses and, to some extent, the reactivation of central memory T cells while it does not influence effector T cells that have fewer costimulatory requirements or use alternative pathways.

The notion of a T cell tolerance defect in RA pathogenesis is supported by genetic studies. RA is a polygenic disease. The strongest genetic risk factor is the shared epitope encoded by HLA-DRB1 alleles, followed by a missense mutation in PTPN22. PTPN22 is a lymphocyte-specific tyrosine phosphatase that is involved in the regulation of signaling cascade after TCR stimulation. The findings of disease-associated HLA-DRB1 alleles and PTPN22 polymorphism indicate that T cell recognition events and TCR threshold calibration may have a role in RA pathogenesis. In addition to the influence of genetic risk factors, signaling rewiring may be acquired, develop overtime and may precede disease onset; indeed, age is one of the strongest risk factors for RA, and the T cell system in RA patients has many signatures that can be explained as aging-related adaptations. See, for example, Weyand et al. (2003) Exp Gerontol 38:833-841.

The development and analysis of biomarkers is of great clinical interest. Such biomarkers could enable the physician to identify patients at risk of developing autoimmune disease, and thus institute early intervention. There is tremendous interest in early intervention, including multiple clinical trials to investigate therapeutic agents in such regimens.

### SUMMARY OF THE INVENTION

Compositions and methods are provided for diagnosis of an autoimmune disease, including without limitation rheumatoid arthritis, diabetes mellitus, multiple sclerosis and the like; or a predisposition to such an autoimmune disease, the method comprising determining the level of expression of one or both of B-Raf and K-Ras in a T cell or T cell population in a sample obtained from an individual, wherein increased expression of one or both of B-Raf and K-Ras, relative to a normal control, in T cells is indicative of a T cell signaling defect leading to a predisposition to autoimmune disease. In some embodiments of the invention, a population of cells comprising T cells is obtained from a patient sample, e.g. a blood sample, and enriched for T cells prior to expression analysis. Therapy may be initiated based on the diagnostic or prognostic findings.

Selective inhibition of one or both of B-Raf and K-Ras is useful in the treatment of autoimmune disease, particularly T cell associated autoimmune disease, which includes, without limitation, rheumatoid arthritis, diabetes mellitus, multiple sclerosis and the like. In such therapeutic methods, a selective inhibitor of B-Raf or K-Ras is administered to an individual suffering from an autoimmune disease, usually an ongoing autoimmune disease, which administration may be localized to the site of lesions, e.g. synovial tissue in RA, or may be systemic. In some embodiments that inhibitor is a selective inhibitor of B-Raf, which inhibitor does not inhibit C-Raf. In preferred embodiments the inhibitor inhibits wild-type B-Raf.

In other embodiments methods are provided for screening of candidate agents for efficacy in the treatment of autoimmune disease, the methods comprising determining the activity of a candidate agent in inhibiting the activity of one or both of B-Raf and K-Ras, for example by inhibition of the kinase activity of the protein or using methods known in the art, and validating a candidate agent for activity in diminishing the effects of an autoimmune disease by administration to a cell or animal model for the disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Expression of ERK pathway genes in RA T cells. Expression of SOS1 (A), RasGRP1 (B), DUSP5 (C) and DUSP6 (D) that regulate the ERK pathway in T cells and are not included in the superarray (Table 3) were quantified by SYBR qPCR. Results from 15 controls (open boxes) and 15 RA patients (shaded boxes) are expressed as the number of transcripts per 10⁶ β-actin transcripts and are shown as box plots.

FIG. 2. Increased expression of K-Ras and B-Raf in RA T cells. Differential expression of K-Ras and B-Raf in the array (Table 3) was confirmed by qPCR, Western blotting and flow cytometry. (A) K-Ras mRNA expression in T cells from 15 control individuals and 15 RA patients was determined by qPCR (top left). A representative Western blot of K-Ras and β-actin is shown in the top right panel. Flow cytometric assessments of K-Ras expression in CD4 (left) and CD8 T cells (right) are shown as representative histograms from a control individual (dashed line) and from an RA patient (bolded line) compared to isotype control antibody (filled histogram) in lane 2; data from 17 patients and 16 controls are summarized in lane 3. Lane 4 shows representative histograms, Lane 5 box plots of N-Ras expression in 20 controls and 18 RA patients. (B) B-Raf transcripts were quantified by qPCR in T cells from 15 controls and 15 RA patients (top left). B-Raf protein expression was quantified by Western blots and by flow cytometry. Representative Western blots (top right) and histograms (lane 2) for CD4 (left) and CD8 T cells (right) from a control (dashed line) and an RA patient (bold line) are shown. Lane 3 summarizes the data from 32 controls and 34 RA patients as box plots. For comparison, representative C-Raf histograms (lane 4) and summarized data from 20 controls and 18 RA patients (lane 5) are shown.

FIG. 3. Linkage of B-Raf and K-Ras expression at the single cell level. T cells from controls and RA patients were stained with anti-CD4, CD8, B-Raf and K-Ras antibodies in parallel. (A) Representative scatter plots of B-Raf versus K-Ras expression in CD4 (left) and CD8 (right) T cells from an RA patient are shown. (B) Cells were gated based on increasing B-Raf fluorescence intensities. MFI of K-Ras and CD4 or CD8 for cells in each of six B-Raf windows was determined. Results are shown as mean±SD of 5 RA patients.

FIG. 4. K-Ras and B-Raf expression is critical for ERK activation in T cells. T cells were transfected with siRNA specific for K-Ras (A) or B-Raf (B); knockdown efficiency was confirmed by Western blot and flow cytometry. Lowered B-Raf or K-Ras expression in T cells from RA patients significantly reduced activation-induced ERK phosphorylation; results are shown as mean±SD of triplicates (C) Cells transfected with K-Ras and B-Raf siRNAs were stimulated with PMA+ ionomycin. MFI of p-ERK1/2 in CD4 and CD8 T cells is shown as mean±SD of triplicates.

FIG. 5. Correlation between B-Raf expression and ERK phosphorylation in RA T cells. T cells from RA patients were stimulated by anti-CD3/CD28 cross-linking and stained with anti-CD 4, CD8, B-Raf and p-ERK1/2 antibodies. (A) Representative scatter plots of B-Raf and p-ERK in CD4 (left) and CD8 (right) T cells at 0, 5 and 10 minutes after stimulation. CD4 and CD8 expression respectively in relationship to B-Raf is shown for comparison. (B) CD4 (left) and CD8 (right) cells were divided into six gates of increasing B-Raf fluorescence intensities. For each window, MFIs of phosphorylated ERK and CD4 or CD8 were determined. Data are shown as mean±SD of 5 RA patients.

FIG. 6. Kinetics of Ras/Raf colocalization in RA T cells. T cells were stimulated by anti-CD3/CD28 crosslinking, fixed, permeablised and stained with Alexa-Fluor 546 conjugated anti-K-Ras (red, left panel) or N-Ras (red, right panel) and Alexa-Fluor 488 conjugated anti-B-Raf (green) antibodies. (A) Representative stainings from a control individual (top lane) and an RA patient (bottom lane) are shown. Co-localization is indicated by yellow. (B) To quantify co-localization, red and green fluorescence for each pixel were correlated. Correlation coefficients are shown as box plots of 75 cells from 5 patients (shaded boxes) and 5 control individuals (open boxes).

FIG. 7. Activation of ERK activation amplification loops in RA T cells. (A) T cell lysates were probed for the presence of phosphorylated compared to total RKIP as indicator of the activation of an amplification loop. Two paired samples of healthy control individuals and RA patients are shown. (B) Cells lysates from anti-CD3/CD28 antibody-stimulated T cells were immunoprecipitated with anti-RKIP antibodies. Precipitates were probed for C-Raf and RKIP by Western blotting. IgG heavy chain is shown as control. The experiments shown are representative of four.

FIG. 8. Functional consequences of increased B-Raf and K-Ras expression. (A) CD4 T cells were transfected with K-RAS or B-RAF. Representative histograms are shown. (B) Transfected cells were stimulated by anti-CD3 or anti-CD3 plus anti-CD28 antibody cross-linking. Proliferation was assessed by 3H-thymidine incorporation, results are shown as mean±SD of quadruplicates and are representative of three experiments. (C) Transfected CD4 T cells from HLA-DRB1*04+ individuals were stimulated with native or citrullinated vimentin peptide for seven days. 3H-thymidine incorporation is shown as mean±SD of quadruplicates and are representative of four experiments.

### DEFINITIONS

Terms used in the claims and specification are defined as set forth below unless otherwise specified.

The term “ameliorating” refers to any therapeutically beneficial result in the treatment of a disease state, e.g., an autoimmune disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

Certain classifications are based on validated criteria, for example overt RA is defined by ACR Criteria (Table 1).

TABLE 1
The 1987 Revised Criteria for the Diagnosis of RA.

| Criterion | Definition |
|---|---|
| Morning | Morning stiffness in and around the joints, |
| stiffness | lasting at least 1 hour before maximal |
| | improvement. |
| Arthritis of 3 | At least three joint areas simultaneously |
| or more joint | with soft tissue swelling or joint fluid |
| areas | observed by a physician; the 14 possible |
| | areas are (right or left): PIP, MCP, wrist, |
| | elbow, knee, ankle, and MTP joints. |
| Arthritis of | At least 1 area swollen in a wrist, MCP, or |
| hand joints | PIP joint |
| Symmetric | Simultaneous involvement of the same |
| arthritis | joint areas on both sides of the body |
| | (bilateral involvement of the PIP, MCP, or |
| | MTP acceptable without perfect |
| | symmetry). |
| Rheumatoid | Subcutaneous nodules over bony |
| nodules | prominences or extensor surfaces, or in |
| | juxtaarticular regions, observed by a |
| | physician. |
| Serum rheumatoid | Abnormal amount of serum RF by any |
| factor | method for which the result has been |
| | positive in <5% of control subjects |
| Radiographic | Erosions or unequivocal bony |
| changes | decalcification localized in or most marked |
| | adjacent to the involved joints |
| | (osteoarthritis changes excluded), typical |
| | of RA on posteroanterior hand and wrist |
| | radiographs. |

For classification purposes, a patient is said to have RA if four of seven criteria are present. Criteria 1-4 must have been present for at least 6 weeks.
*Taken from Arnett F C, Edworthy S M, Bloch D A, McShane D J, Fries J F, Cooper N S, Healey L A, Kaplan S R, Liang M H, Luthra H S, et al. The American Rheumatism Association 1987 revised criteria for the classification of rheumatoid arthritis. Arthritis Rheum 1988; 31: 315-324.

Analysis of results may utilize the common meaning certain terms, including:

TP: true positive
TN: true negative
FP: false positive
FN: false negative
N: total number of negative samples
P: total number of positive samples
A: total number of samples

Accuracy=(TP+TN)/A

Mean CV error=Mean Misclassification error=1−Mean Accuracy

Sensitivity=TP/P=TP/(TP+FN)

Specificity=TN/N=TN/(TN+FP)

It must be noted that, as used in the specification and the appended claims, the singular forms “a,” “an,” and “the” include plural referents unless the context clearly dictates otherwise.

B-Raf is a 651-amino acid protein with a calculated molecular mass of 72.5 kD. It contains all 3 conserved regions of RAF protein kinases: a putative zinc finger region, a serine/threonine-rich region, and a C-terminal kinase domain, which includes a putative ATP-binding site and a catalytic lysine. In addition, the N terminus of BRAF is serine rich, and it has a consensus CDC2 phosphorylation motif. The sequence of the human protein and gene is available at Genbank, accession number EU600171, herein specifically incorporated by reference. Mutations in B-Raf are frequently found in cancers, with the most common being the V600E mutation.

K-Ras is a GDP/GTP-binding protein that acts as an intracellular signal transducer. The KRAS2 gene encodes a 188-residue protein with a molecular mass of 21.66 kD. The sequence of the human protein and gene is available at Genbank, accession number EU332849, herein specifically incorporated by reference. Comparison of the 2 KRAS genes showed that KRAS1 is a pseudogene derived from a processed KRAS2 mRNA, while KRAS2 is the active human gene. Alternative splicing results in 2 variants, isoforms A and B, that differ in the C-terminal region. The differing C-terminal regions of these isoforms are subjected to posttranslational modifications having functional effects leading to alternative trafficking pathways and protein localization.

Inhibitory agents: an agent that inhibits the activity of the target protein, e.g. B-Raf and/or K-Ras. The inhibitory agent may inhibit the activity of the target protein by a variety of different mechanisms. In certain embodiments, the inhibitory agent is one that binds to the target protein and, in doing so, inhibits its activity. A number of B-Raf inhibitors are known in the art, although many are undesirably selective for the V600E BRAF mutation. Among inhibitors useful for the methods of the invention are Sorafenib and derivatives thereof; and inhibitors set forth, for example, in Carnahan et al. (2010) Mol Cancer Ther. 9(8):2399-410; Blackburn et al. (2010) Bioorg Med Chem. Lett. 20(16):4795-9; Pratilas et al. (2010) Clin Cancer Res. 16(13):3329-34; Smith et al. (2009) J Med. Chem. 52(20):6189-92; Xie et al. (2009) Biochemistry. 48(23):5187-98; Wong et al. (2009) J Pharmacol Exp Ther. 329(1):360-7; Takle et al (2008) Bioorg Med Chem. Lett. 18(15):4373-6; Takle et al. (2006) Bioorg Med Chem. Lett. 16(2):378-81.

In other embodiments, the inhibitory agent alters the expression of the protein through interference with transcription or translation of the encoding gene. Representative genetic inhibitory agents include, but are not limited to: antisense oligonucleotides, RNAi, and the like. Other agents of interest include, but are not limited to naturally occurring or synthetic small molecule compounds of interest, which include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing appropriate screening protocols.

The antisense reagent may be antisense oligonucleotides (ODN), particularly synthetic ODN having chemical modifications from native nucleic acids, or nucleic acid constructs that express such antisense molecules as RNA. The antisense sequence is complementary to the targeted coding sequence, and inhibits its expression. One or a combination of antisense molecules may be administered, where a combination may comprise multiple different sequences.

Antisense molecules may be produced by expression of all or a part of the target protein sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the mRNA encoding the targeted protein. By RNAi agent is meant an agent that modulates expression of an mRNA by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

### DETAILED DESCRIPTION OF THE EMBODIMENTS

Compositions and methods are provided for prognostic classification of autoimmune disease patients, which prognosis is informative of the patient's probability of developing overt disease. The patterns of expression of B-Raf and/or K-Ras in T cells from an individual provides for a signature pattern that can discriminate patients who have a high probability of developing autoimmune disease from those who have a low probability of developing autoimmune disease. The signature pattern in a patient has predictive and diagnostic value, and thus allows improved methods of care, where patients can be provided with appropriate therapy. In one embodiment of the invention, the autoimmune disease is rheumatoid arthritis.

Various techniques and reagents find use in the diagnostic methods of the present invention. In one embodiment of the invention, a patient cellular sample such as a blood sample, CNS fluid, synovial fluid, etc. is assayed for the expression of specific sequences in activated T cells. Typically a blood sample is drawn, and a cellular product, such as a population of peripheral blood lymphocytes, selected T cell population, etc. is analyzed by any convenient method for expression of the selected sequences, and compared to a known normal control.

Mammalian species that provide samples for analysis include canines; felines; equines; bovines; ovines; etc. and primates, particularly humans. Animal models, particularly small mammals, e.g. murine, lagomorpha, etc. may be used for experimental investigations. Animal models of interest include those for models of autoimmunity and the like.

In addition to the specific biomarker sequences identified in this application by name, accession number, or sequence, the invention also contemplates use of biomarker variants that are at least 90% or at least 95% or at least 97% identical to the exemplified sequences and that are now known or later discover and that have utility for the methods of the invention. These variants may represent polymorphisms, splice variants, mutations, and the like. Various techniques and reagents find use in the diagnostic methods of the present invention. In one embodiment of the invention, blood samples, or samples derived from blood, e.g. plasma, circulating, etc. are assayed for the presence of polypeptides. Typically a blood or lymph sample is drawn, and a derivative product, such as a preparation of peripheral blood monocytes, enriched T cell population, etc., is tested. Such a cell population is analyzed for the presence of increased levels of protein or mRNA corresponding to the biomarkers identified herein. Various formats find use for such assays, including antibody arrays; ELISA and RIA formats; binding of labeled antibodies in suspension/solution and detection by flow cytometry, mass spectroscopy, and the like; or for detection of mRNA a variety of amplification and/or hybridization assays may find use. Expression signatures typically utilize a detection method coupled with analysis of the results to determine if there is a statistically significant match with a disease signature, i.e. an upregulation in expression of B-Raf and/or K-ras relative to the expression level in normal T cells.

### Conditions for Analysis and Therapy

The compositions and methods of the invention find use in combination with a variety of autoimmune conditions. Of particular interest is rheumatoid arthritis, which is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Additional non-HLA genes are also associated with increased risk for disease including polymorphisms in PTPN22 and CTLA4 loci. Environmental factors may also play a role. Immunologic changes may be initiated by multiple factors. About 0.6% of all populations are affected, women two to three times more often than men. Onset may be at any age frequently after the age of 50 years.

In over 80% of cases, RA is characterized by the presence of the autoantibodies rheumatoid factor (RF) and antibodies directed against citrullinated peptides, of which the antibody to a cyclic citrullinated peptide (anti-CCP antibody) is most clinically useful. These antibodies have been shown to be present years prior to the onset of symptoms in RA, suggesting that the immunologic processes that lead to disease are present long before overt disease manifestations (Nielen M M, van Schaardenburg D, Reesink H W, van de Stadt R J, van der Horst-Bruinsma I E, de Koning M H, et al. Specific autoantibodies precede the symptoms of rheumatoid arthritis: a study of serial measurements in blood donors. Arthritis Rheum. 2004 February; 50(2):380-6; Rantapaa-Dahlqvist S, de Jong B A, Berglin E, Hallmans G, Wadell G, Stenlund H, et al. Antibodies against cyclic citrullinated peptide and IgA rheumatoid factor predict the development of rheumatoid arthritis. Arthritis Rheum. 2003 October; 48(10):2741-9. Also, in additional pre-clinical studies the presence of specific HLA DR4 alleles along with RA-specific antibodies was highly predictive of eventual development of symptomatic RA (Berglin E, Padyukov L, Sundin U, Hallmans G, Stenlund H, Van Venrooij W J, Klareskog L, Dahlqvist S R. A combination of autoantibodies to cyclic citrullinated peptide (CCP) and HLA-DRB1 locus antigens is strongly associated with future onset of rheumatoid arthritis. Arthritis Res Ther. 2004; 6(4):R303-8. Epub 2004 May 11). RA that does not exhibit autoantibodies is termed sero-negative RA and comprises less than 20% of the total cases. This variation of disease represents a distinct subset that appears both immunolgically and clinically different than sero-positive RA.

Prominent immunologic abnormalities that may be important in pathogenesis include immune complexes found in joint fluid cells and in vasculitic lesions. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease. Effector molecules such as complement activation fragments are released into the tissue and joint fluid and can cause both direct and indirect injury. Engagement of activating Fc receptors by immune complexes can potentiate injury to the joint.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; interleukin-1, which stimulates lymphocyte proliferation; and prostaglandins. The infiltrating cells, initially perivenular but later forming lymphoid follicles with germinal centers, synthesize interleukin-2, other cytokines, RF, and other immunoglobulins. Fibrin deposition, fibrosis, and necrosis also are present. Hyperplastic synovial tissue (pannus) may erode cartilage, subchondral bone, articular capsule, and ligaments. PMNs are not prominent in the synovium but often predominate in the synovial fluid.

Onset of clinically apparent disease is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

In RA, response to therapy is conventionally measured using the American College of Rheumatology (ACR) Criteria. The ACR response criteria are a composite score comprising clinical (swollen joint count, tender joint count, physician and patient response assessment, and health assessment questionnaire), and laboratory (acute phase response) parameters; level of improvement is reported as an ACR20 (20%), ACR50 (50%) or ACR70 (70%) response, which indicates percent change (improvement) from the baseline score. A number of clinical trials based on which the anti-TNFα agents infliximab (Remicade™), etanercept (Enbrel™) and adalimumab (Humira™) were approved to treat human RA utilized ACR response rates as a primary outcome measure.

Responses in rheumatoid arthritis many also be assessed using other response criteria, such as the Disease Activity Score (DAS), which takes into account both the degree of improvement and the patient's current situation. The DAS has been shown to be comparable in validity to the ACR response criteria in clinical trials. The definitions of satisfactory and unsatisfactory response, in accordance with the original DAS and DAS28. The DAS28 is an index consisting of a 28 tender joint count, a 28 swollen joint count, ESR (or CRP), and an optional general health assessment on a visual analogue scale (range 0-100) (Clinical and Experimental Rheumatology, 23(Suppl. 39):593-99, 2005). DAS28 scores are being used for quantification of response mostly in European trials of (early) rheumatoid arthritis such as the COBRA or BeST studies (described below). In asymptomatic individuals predicted to develop RA, response could be assessed based on progression to clinically definite RA based on the 1987 Revised Criteria for the Diagnosis of RA (Table 1).

Radiographic measures for response in RA include both conventional X-rays (plain films), and more recently magnetic resonance (MR) imaging, computed tomography (CT), ultrasound and other imaging modalities are being utilized to monitor RA patients for disease progression. Such techniques are used to evaluate patients for inflammatoin (synovitis), joint effusions, cartilage damage, bony erosions and other evidence of joint damage. Methotrexate, anti-TNF agents and DMARD combinations have been demonstrated to reduce development of bony erosions and other measures of joint inflammation and destruction in RA patients. In certain cases, such as with anti-TNF agents, healing of bony erosions has been observed.

### Diagnostic and Prognostic Methods

The differential expression of B-Raf and/or K-Ras in T cells provides prognostic evaluations to detect individuals in a pre-disease state for an autoimmune disease. In general, such prognostic methods involve determining the presence or level of the protein or corresponding mRNA in an individual sample, usually a blood or lymph derived sample, e.g. PBMC preparation, cell populations enriched for CD3+ cells, cell populations enriched for CD4+ T cells, etc. A variety of different assays can be utilized to quantitate the presence of these biomarkers.

An RNA sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a T cell population from a subject to be diagnosed, using standard protocols, where cell types or tissues from which such nucleic acids may be generated include any tissue in which the expression pattern of the to be determined phenotype exists. Cells may be cultured, e.g. activated by antigen, prior to analysis.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Alternatively, non-array based methods for quantitating the levels of one or more nucleic acids in a sample may be employed, including quantitative PCR, and the like.

Where the expression profile is a protein expression profile, any convenient protein quantitation protocol may be employed, where the levels of one or more proteins in the assayed sample are determined. Many such methods are known to one of skill in the art, including ELISA, fluorescence immunoassays, protein arrays, eTag system, bead based systems, tag or other array based systems, surface plasmon resonance (SPR)-based detection systems, etc. Examples of such methods are set forth in the art, including, inter alia, chip-based capillary electrophoresis: Colyer et al. (1997) J Chromatogr A. 781(1-2):271-6; mass spectroscopy: Petricoin et al. (2002) Lancet 359: 572-77; eTag systems: Chan-Hui et al. (2004) Clinical Immunology 111:162-174; microparticle-enhanced nephelometric immunoassay: Montagne et al. (1992) Eur J Clin Chem Clin Biochem. 30(4):217-22; the Luminex XMAP bead array system; and the like, each of which are herein incorporated by reference.

Following obtainment of the expression profile from the sample being assayed, the expression profile is compared with a reference or control profile to make a diagnosis. A reference or control profile is provided, or may be obtained by empirical methods from samples of normal T cells. In certain embodiments, the obtained expression profile is compared to a single reference/control profile to obtain information regarding the phenotype of the sample being assayed. In yet other embodiments, the obtained expression profile is compared to two or more different reference/control profiles to obtain more in depth information regarding the phenotype of the assayed sample. For example, the obtained expression profile may be compared to a positive and negative reference profile to obtain confirmed information regarding whether the sample has the phenotype of interest. The readout may be a mean, average, median or the variance or other statistically or mathematically-derived value associated with the measurement.

Samples can be obtained from the tissues or fluids of an individual. For example, samples can be obtained from whole blood, tissue biopsy, synovial fluid, lymph, cerebrospinal fluid, bronchial aspirates, and the like. Also included in the term are derivative populations and fractions of such cells. Diagnostic samples are collected any time after an individual is suspected to have a predisposition to an autoimmune disease, has exhibited symptoms that predict onset of autoimmune disease, or have exhibited symptoms of an autoimmune disease.

Also provided are reagents and kits thereof for practicing one or more of the above-described methods. The subject reagents and kits thereof may vary greatly. Reagents of interest include reagents specifically designed for use in production of the above described expression profiles of T cell markers associated with autoimmune conditions.

One type of such reagent is an array or kit of antibodies that bind to a marker set of interest. A variety of different array formats are known in the art, with a wide variety of different probe structures, substrate compositions and attachment technologies. Representative array or kit compositions of interest include or consist of reagents for quantitation of one or both of B-Raf and K-Ras, and may include reagents for enrichment of T cells from a mixed cell population

The kits may further include a software package for statistical analysis of one or more phenotypes, and may include a reference database for calculating the probability of classification. The kit may include reagents employed in the various methods, such as devices for withdrawing and handling blood samples, second stage antibodies, ELISA reagents; tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Therapeutic Agents

In one embodiment of the invention, inhibitors of B-Raf and/or K-Ras activity, e.g. anti-sense oligonucleotides, RNAi, small molecule inhibitors of protein activity and the like are used in the treatment of T cell mediated autoimmune disease, including rheumatoid arthritis, MS, IDDM, etc.

The method also provide for combination therapy, where the combination may provide for additive or synergistic benefits. Combinations of a B-Raf and/or K-Ras inhibitor may be obtained with a second agent selected from one or more of the general classes of drugs commonly used in the non-antigen specific treatment of autoimmune disease, which include corticosteroids and disease modifying drugs; or from an antigen-specific agent. Corticosteroids have a short onset of action, but many disease modifying drugs take several weeks or months to demonstrate a clinical effect. These agents include methotrexate, leflunomide (Arava™) etanercept (Enbrel™), infliximab (Remicade™), adalimumab (Humira™), anakinra (Kineret™) rituximab (Rituxan™), CTLA4-Ig (abatacept), antimalarials, gold salts, sulfasalazine, d-penicillamine, cyclosporin A, cyclophosphamide azathioprine; and the like.

Corticosteroids, e.g. prednisone, methylpredisone, prednisolone, solumedrol, etc. have both anti-inflammatory and immunoregulatory activity. They can be given systemically or can be injected locally. Corticosteroids are useful in early disease as temporary adjunctive therapy while waiting for disease modifying agents to exert their effects. Corticosteroids are also useful as chronic adjunctive therapy in patients with severe disease.

Disease modifying anti-rheumatoid drugs, or DMARDs have been shown to alter the disease course and improve radiographic outcomes in RA. It will be understood by those of skill in the art that these drugs are also used in the treatment of other autoimmune diseases.

Methotrexate (MTX) is a frequent first-line agent because of its early onset of action (4-6 weeks), good efficacy, favorable toxicity profile, ease of administration, and low cost. MTX is the only conventional DMARD agent in which the majority of patients continue on therapy after 5 years. MTX is effective in reducing the signs and symptoms of RA, as well as slowing or halting radiographic damage. Although the immunosuppressive and cytotoxic effects of MTX are in part due to the inhibition of dihydrofolate reductase, the anti-inflammatory effects in rheumatoid arthritis appear to be related at least in part to interruption of adenosine and TNF pathways. The onset of action is 4 to 6 weeks, with 70% of patients having some response. A trial of 3 to 6 months is suggested.

Inhibitors as described herein can serve as the active ingredient in pharmaceutical compositions formulated for the treatment of various disorders as described above. The active ingredient is present in a therapeutically effective amount, i.e., an amount sufficient when administered to substantially modulate the effect of the targeted protein or polypeptide to treat a disease or medical condition mediated thereby. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

When the pharmaceutical composition includes a polypeptide as the active ingredient, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which are composed of the packaged active ingredient with a suppository base. Suitable suppository bases include natural or synthetic triglycerides or paraffin hydrocarbons. In addition, it is also possible to use gelatin rectal capsules, which are composed of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### Methods of Treatment

The inhibitor compositions may be administered in a single dose, or in multiple doses, usually multiple doses over a period of time, e.g. daily, every-other day, weekly, semi-weekly, monthly etc. for a period of time sufficient to reduce severity of the inflammatory disease, which may comprise 1, 2, 3, 4, 6, 10, or more doses.

Determining a therapeutically or prophylactically effective amount an agent that provides sufficient inhibitor activity can be done based on animal data using routine computational methods. In one embodiment, the therapeutically or prophylactically effective amount contains between about 0.01 mg and about 100 mg of active agent, as applicable. The effective dose will depend at least in part on the route of administration. The agents may be administered orally, in an aerosol spray; by injection, e.g. i.m., s.c., i.p., i.v., etc.

The inhibitor compositions are administered in a pharmaceutically acceptable excipient. The term “pharmaceutically acceptable” refers to an excipient acceptable for use in the pharmaceutical and veterinary arts, which is not toxic or otherwise inacceptable. The concentration of αBC compositions of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Treating, treatment, or therapy of a disease or disorder shall mean slowing, stopping or reversing the disease's progression by administration of an inhibitor of the invention. In the preferred embodiment, treating a disease means reversing the disease's progression, ideally to the point of eliminating the disease itself. As used herein, ameliorating a disease and treating a disease are equivalent. Preventing, prophylaxis or prevention of a disease or disorder as used in the context of this invention refers to the administration of an inhibitor composition to prevent the occurrence or onset of a disease or disorder or some or all of the symptoms of a disease or disorder or to lessen the likelihood of the onset of a disease or disorder.

Biomarkers that facilitate identification of asymptomatic individuals likely to develop an autoimmune disease, or early symptomatic individuals likely to progress to an autoimmune disease, facilitate implementation of therapies that reduce the ultimate incidence and/or severity of clinically active disease. Such biomarkers may identify patients likely to experience a mild disease course and thus warrant less aggressive therapy from patients likely to develop severe disease and thus warrant more aggressive therapy. Further, such biomarkers may identify patients likely to respond to specific therapeutic agents, and could thereby be used to guide selection of the most appropriate agent(s) to treat individual patients that are asymptomatic (pre-disease), exhibiting early symptoms, or have established disease.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference.

### EXPERIMENTAL

### Example 1

The findings of disease-associated HLA-DRB1 alleles and PTPN22 polymorphism indicate that T cell recognition events and TCR threshold calibration are central to RA pathogenesis. The majority of RA patients should have TCR signal processing abnormalities that have been acquired, because the PTPN22 polymorphism is only present in less than one quarter of RA patients. Such signaling rewiring may develop overtime and may precede disease onset; indeed, age is one of the strongest risk factors for RA, and the T cell system in RA patients has many signatures that can be explained as aging-related adaptations.

To examine this hypothesis, we have compared the signaling potential after TCR stimulation in RA patients and age-matched healthy subjects and have found a hyperactive Ras/Raf-MEK-ERK module. The underlying abnormality is an overexpression of B-Raf and K-Ras. Expression of B-Raf, which is limiting in normal T cells, overcomes anergy if expressed at higher concentration. We demonstrate that this mechanism is causing the tolerance defects in RA.

### Materials and Methods

Study Population and Cells: T cells were isolated by negative selection using RosetteSep human T-cell enrichment cocktail (StemCell Tech., Vancouver, Canada) from the peripheral blood of RA patients and demographically matched healthy controls. RA patients met the 1988 American College of Rheumatology Criteria for seropositive RA (Table 1). The protocol was approved by the Emory and Stanford University Institutional Review Boards, and all donors gave written, informed consent.

TABLE 1
Demographic characteristics of RA patients and HC populations

| | RA | HC | p |
|---|---|---|---|
| n | 104 | 90 | |
| Age (years) | 50.6 +/− 9.4 | 48.3 +/− 10.1 | 0.111 |
| Gender (F/M) | 81/23 | 73/17 | 0.580 |
| Ethnicity | 51/43/6/4 | 51/33/2/4 | 0.509 |
| (AA/C/H/A)^{a} | | | |

^{a}AA, African American; C, Caucasian; H, Hispanic; A, Asian.

Antibodies: CD3-APC Cy7, CD4-PerCP, CD8-PE Cy7, Alexa Fluor 647-conjugated anti-phospho-ERK1/2 antibodies were from BD Biosciences, San Jose, Calif. In addition, the following antibodies were used: anti-ERK1/2-FITC (Millipore, Billerica, Mass.), antibodies to N-Ras, K-Ras, C-Raf, p-RKIP, RKIP, actin (Santa Cruz Biotech, Santa Cruz, Calif.); anti-phospho-B-Raf and B-Raf (Cell Signaling Technology, Beverly, Mass.). Zenon antibody labeling kits with Alexa Fluor 488, 546 and 647 (Invitrogen, Eugene, Oreg.) were used to conjugate primary antibodies.

Flow cytometry: T cells (1×10⁶) were stimulated or not with anti-CD3/CD28 mAb (1 μg/ml each); fixed in BD Cytofix buffer; permeablized by BD Perm Buffer II or 100% methanol; and stained for CD3, CD4, CD8 and the indicated signaling molecules. Data were acquired and analyzed on an LSR II flow cytometer (BD Biosciences) with FACS DIVA software.

Western Blotting and Immunoprecipitation: T cells were lysed in a cell extraction buffer (Invitrogen) supplemented with 1 mM PMSF and a protease inhibitor cocktail (Sigma-Aldrich, St Louis, Mo.). Thirty micrograms of total protein were resolved on SDS-PAGE under reducing conditions, transferred to PVDF membranes, and incubated at 4° C. with antibodies to anti-phospho-B-Raf, B-Raf or K-Ras followed by washing and incubation with HRP-conjugated secondary antibodies (Santa Cruz Biotechnology). The blots were visualized with an Immobilon Western chemiluminescence detection system (Millipore). Membranes were stripped and re-probed for actin to ensure equal loading. For immunoprecipitation, 200 μg of total cell lysates from anti-CD3/CD28 stimulated T cells were incubated with anti-RKIP antibody overnight at 4° C. followed by washing and Western blot analysis for B-Raf, C-Raf and RKIP. IgG heavy chain was included as a control.

Confocal Microscopy: T cells from control and RA patients stimulated and fixed as described above were stained with Alexa Fluor 546-labeled anti-K-Ras or anti-N-Ras antibodies and Alexa Fluor 488-labeled anti-B-Raf antibody; images were captured by Zeiss confocal laser scanning LSM 510 META Axiovert-200 microscope. Ras-Raf colocalization was quantified by using Image J software (NIH, Bethesda, Md.).

Super array and qPCR: Total RNA isolated from RA and healthy control T cells using Trizol (Invitrogen) was reverse transcribed into cDNA using AMV Reverse Transcriptase (Roche Diagnostic Corp., Indianapolis, Ind.). Quantification of mRNA levels of components of ERK pathway, as well as ERK-dependent genes, was done by MAP Kinase Signaling Pathway PCR Array (SA Biosciences, Frederick, Md.) as per the manufacturer's instructions. In addition, SOS1, RasGRP1, DUSP5, DUSP6, K-Ras and B-Raf transcription was quantified by SYBR quantitative PCR using the following primer sets: SOS1: 5′-ACCACGAGAACCTGTGAG-3′,5′-GAAGGGCTGTTTGGGAAG-3′; RasGRP1: 5′-GCCTTGGATTGGCAGTGA-3′, 5′-GGTAGGCAGTCTGAGGTGA-3′; DUSP5: 5′-CTGAGTGTTGCGTGGATGTA-3′, 5′-TCGCACTTGGATGCATGGTA-3′; DUSP6: 5′-CAGTGGTGCTCTACGACGAG-3′, 5′-GCAATGCAGGGAGAACTCGGC-3′; β-actin: 5′-ATGGCCACGGCTGCTTCCAGC-3′, 5′-CATGGTGGTGCCGCCAGACAG-3′. The PCR conditions for all amplifications were 95° C. for 10 min, followed by 40 cycles of 95° C. for 15 sec, 60° C. for 30 sec, 72° C. for 30 sec. Results are given as transcript numbers determined by interpolation with standard curves using gene-expressing plasmids relative to β-actin transcript numbers.

Gene Silencing: siRNA for K-Ras (Hs_KRAS2_{—}8 HP GenomeWide siRNA) and B-Raf (Hs_BRAF_{—}1 HP GenomeWide siRNA), was obtained from QIAGEN. siRNA oligonucleotides (1.5 μg) were transfected into resting CD3 T cells using Amaxa Nucleofector system and Human T cell Nucleofector kit (Lonza Walkersville Inc., Walkersville, Md.). Negative Control siRNA (QIAGEN) was used as a negative control. Forty-eight to 72 h after transfection, knockdown efficiencies were monitored by flow cytometry and Western blotting.

Transfection: Human K-Ras and B-Raf cDNA were obtained from Open Biosystems (Clone ID are 3878884 and 8327547). Complete open reading frames were inserted into the pIRES2-AcGFP1 empty vector (Clontech). Total CD4 T cells were transfected with empty vector, K-Ras-pIRES2-AcGFP1 or B-Raf-pIRES2-AcGFP1 (4 μg each) using the Amaxa Nucleofector system and the Human T cell Nucleofector kit (Lonza).

### Results

Molecular mechanisms of increased ERK activation in RA T cells. Increased ERK phosphorylation in T cells from RA patients compared to healthy controls was previously reported (Singh et al. (2009) J Immunol 183:8258-8267). It is shown herein that differential transcription of B-Raf and K-Ras explains the increased responsiveness of RA T cells to stimulation. PhosFlow data from a cohort of 65 RA patients and 54 healthy controls documented a significantly higher basal ERK phosphorylation in RA CD4 and CD8 T cells (p<0.0001) while no difference was seen for total ERK (Table 2). After CD3/CD28 cross-linking, the differences in phospho-ERK between RA and control T cells widened, and increased ERK phosphorylation was seen for RA CD4 and CD8 T cells at all time points examined (p<0.0001). CD3 expression was used as a system control to exclude flow artifacts or higher expression of the CD3/TCR complex; no difference was seen.

TABLE 2
Increased activation of the ERK pathway in T cells from RA patients after
TCR stimulation

| | CD4 T cells | | | CD8 T cells | | |
|---|---|---|---|---|---|---|
| | HC | RA | | HC | RA | |
| | Means | Means | | Means | Means | |
| | (SD) | (SD) | p-value | (SD) | (SD) | p-value |
| CD3 | 24311 | 23204 | 0.552 | 19349 | 17379 | 0.125 |
| | (5380) | (5670) | | (3651) | (3850) | |
| Total | 2433 | 2436 | 0.993 | 2497 | 2656 | 0.657 |
| ERK | (1211) | (1100) | | (1100) | (1031) | |
| p- | 403 | 504 | <0.0001 | 475 | 644 | <0.0001 |
| ERK, | (71) | (92) | | (88) | (152) | |
| 0 min | | | | | | |
| p- | 1114 | 1501 | <0.0001 | 1751 | 2512 | <0.0001 |
| ERK, | (263) | (447) | | (446) | (923) | |
| 5 min | | | | | | |
| p- | 813 | 1021 | <0.0001 | 1236 | 1947 | <0.0001 |
| ERK, | (177) | (222) | | (327) | (586) | |
| 10 | | | | | | |
| min | | | | | | |
| p- | 504 | 632 | <0.0001 | 798 | 1168 | <0.0001 |
| ERK, | (90) | (109) | | (177) | (394) | |
| 30 | | | | | | |
| min | | | | | | |

Increased responsiveness of the ERK pathway in RA T cells.
T cells from control individuals and RA patients were stimulated by anti-CD3/CD28 antibody crosslinking.
CD3, total ERK and phosphorylated ERK (pERK) expression in CD4 and CD8 T cells before and at indicated time points after stimulation was determined by flow cytometry.
Data are given as mean ± SD MFI.

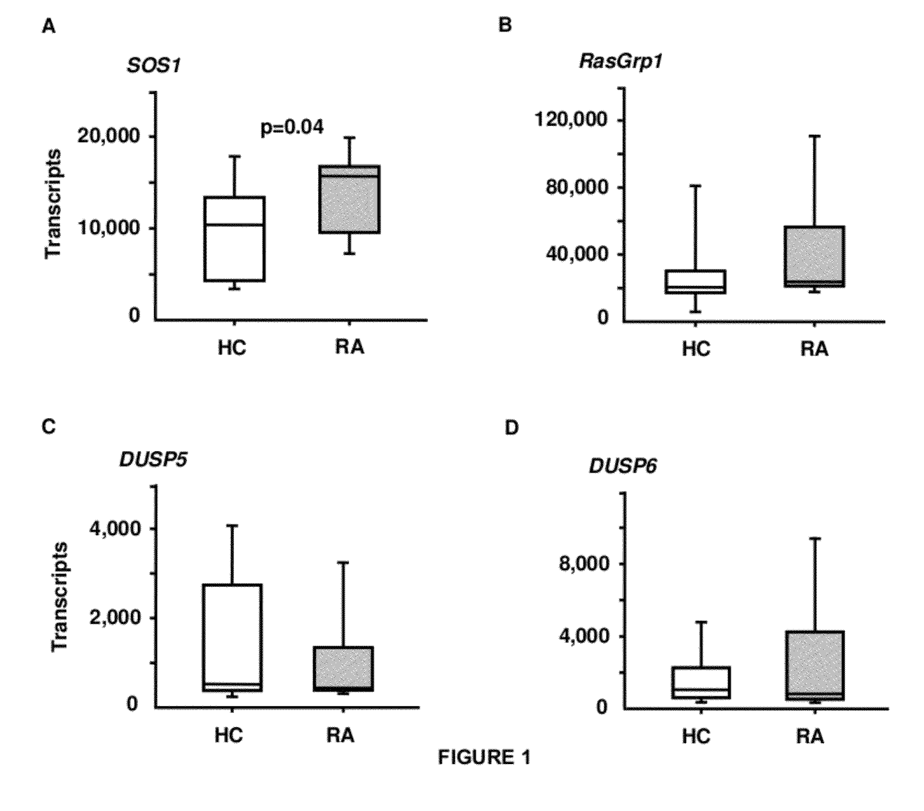

We used a gene expression array (MAP Kinase Signaling Pathway PCR Array from SA Biosciences) to screen for abnormal transcription of ERK pathway constituents in RA T cells. Transcripts with an increase or decrease of more than 1.5-fold were considered candidate genes. The array also included ERK targets genes, many of which were found to be overexpressed consistent with the observed increased phosphorylation of ERK at baseline (Table 3). Of the signaling members of the ERK pathway present in the array, K-Ras, B-Raf and MEK1 were transcribed at higher levels (1.73-fold, 2.05-fold and 1.55-fold, respectively) in RA as compared to control T cells (Table 3) while no difference was seen for the majority of the ERK pathway components. The array did not include several important components of the ERK pathway which were therefore quantified by qPCR. Initial Ras-Raf association after TCR stimulation is controlled by RasGRP1 and SOS-1 (Roose et al. (2007) Mol Cell Biol. 27(7):2732-2745). qPCR studies showed a slightly higher SOS-1 level in RA patients (p=0.04) and no significant difference for RasGRP1 (FIGS. 1A and B). DUSP5 and DUSP6 are the two phosphatases that are expressed in non-activated T cells and that dephosphorylate pERK (19). No difference was seen at the transcriptional level (FIGS. 1C and D).

TABLE 3
Expression of ERK-dependent and ERK pathway genes
in RA patients and healthy controls (HC).

| ERK | RA/HC | ERK | RA/HC |
|---|---|---|---|
| dependent genes | (Fold difference) | pathway genes | (Fold difference) |
| CCNA1 | 3.28 | GRB2 | 1.04 |
| CCNA2 | 1.74 | H-RAS | −1.32 |
| CCNB1 | 1.98 | K-RAS | 1.73 |
| CCNB2 | 2.48 | N-RAS | −1.05 |
| CDK6 | 1.50 | KSR1 | 1.30 |
| CDKN1C | 2.19 | A-RAF | −1.12 |
| CDKN2D | 1.54 | B-RAF | 2.05 |
| EGFR | 1.82 | C-RAF | 1.01 |
| ETS2 | 2.22 | MEK1 | 1.55 |
| FOS | 1.77 | MEK2 | −1.45 |
| MEF2C | 1.71 | ERK1 | −1.40 |
| SMAD4 | 1.91 | ERK2 | −1.41 |

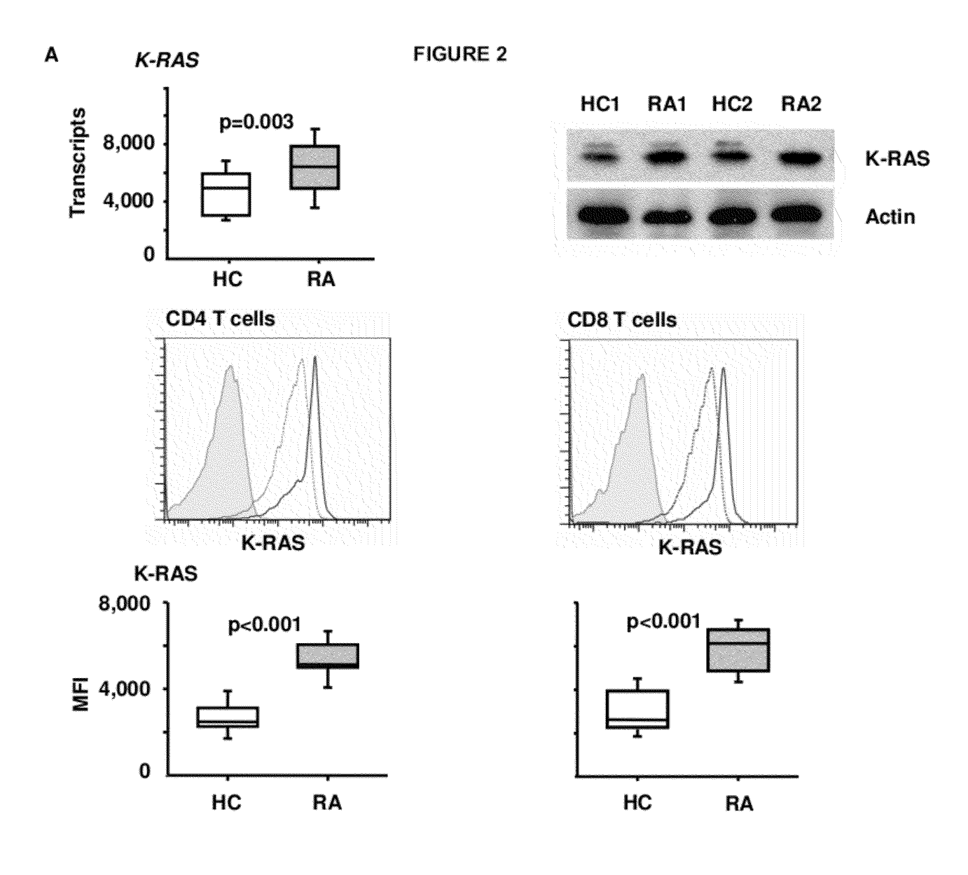

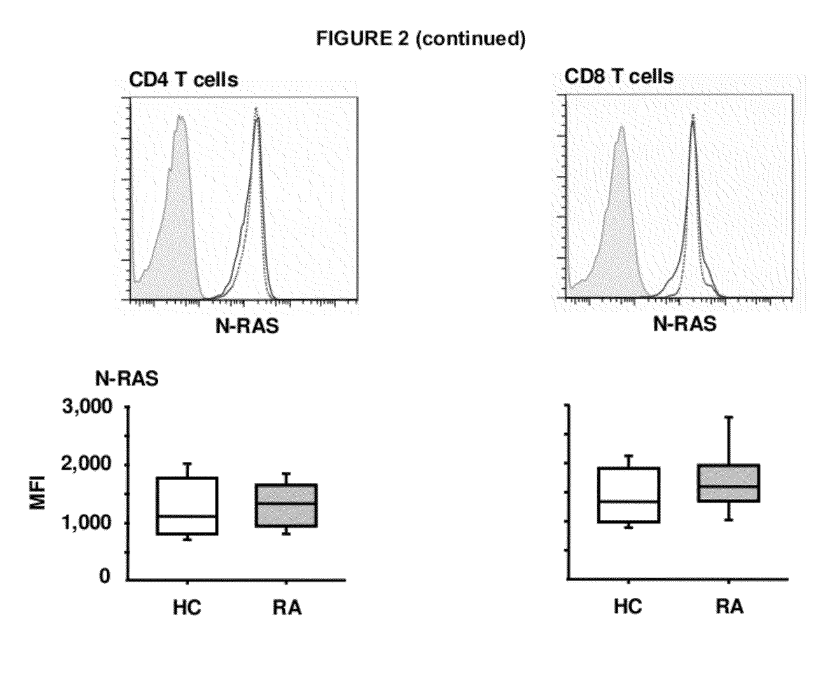

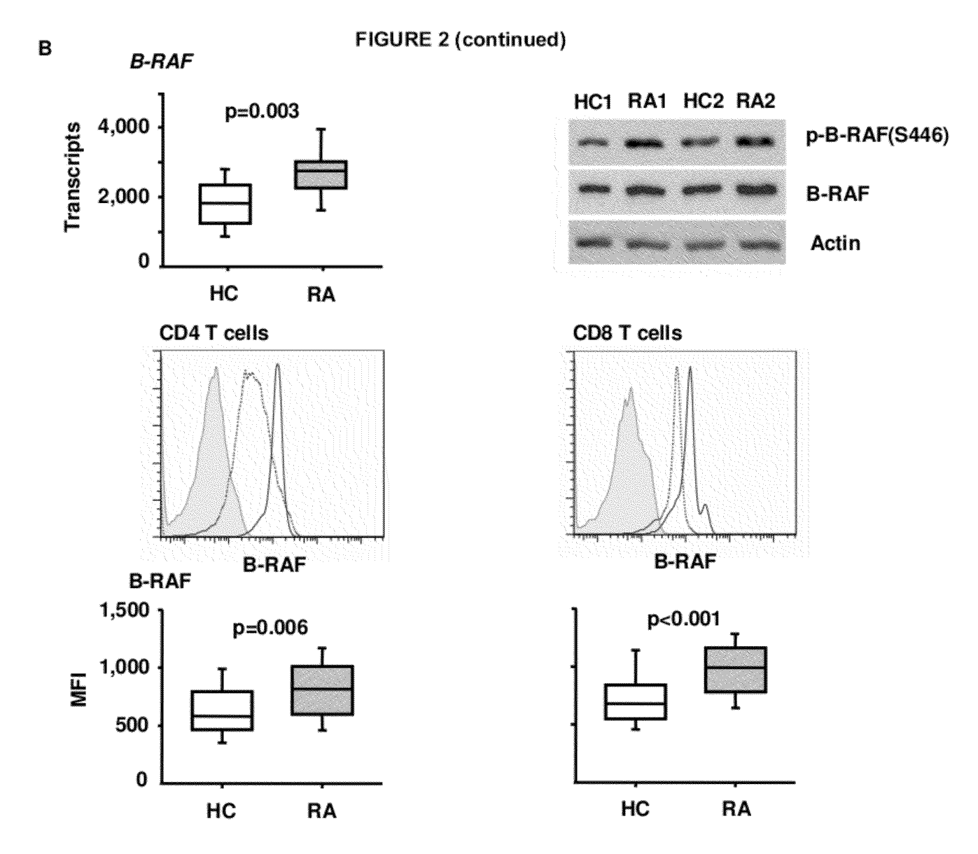

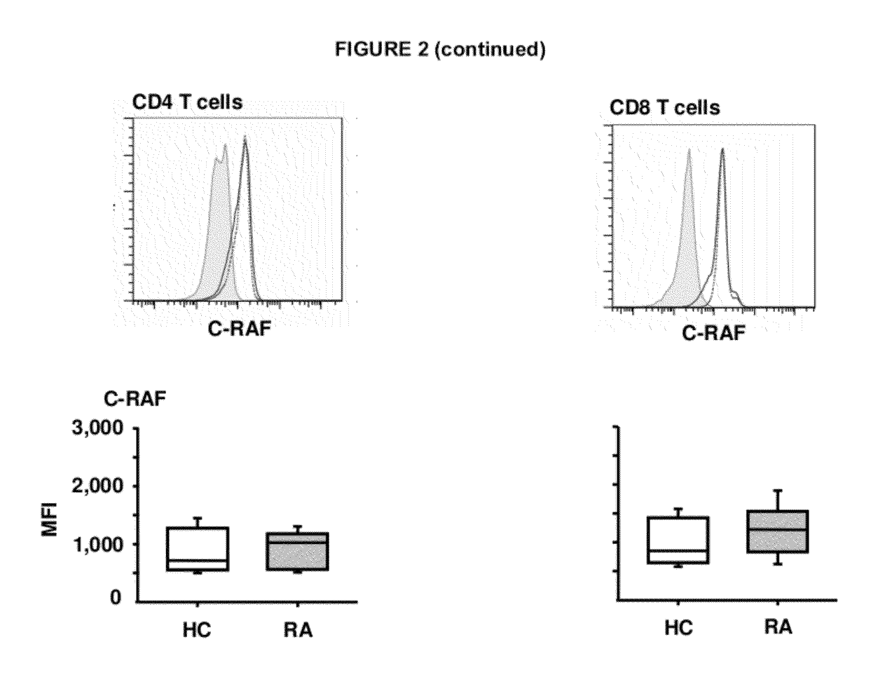

In the following, we focused on those molecules that were most convincingly overexpressed in the screening assays, B-Raf and K-Ras. Overexpression was confirmed by qPCR at the transcriptional level and Western blots and flow cytometry at the protein level (FIG. 2). K-Ras (p=0.003) and B-Raf transcripts (p=0.003) were significantly increased in RA T cells. In the flow cytometric studies, N-Ras and C-Raf not differentially expressed in the array were stained as controls. RA T cells expressed significantly higher levels of K-Ras (p<0.001) and B-Raf (p=0.006 for CD4, p<0.001 for CD8 T cells) while having similar levels of N-Ras and C-Raf.

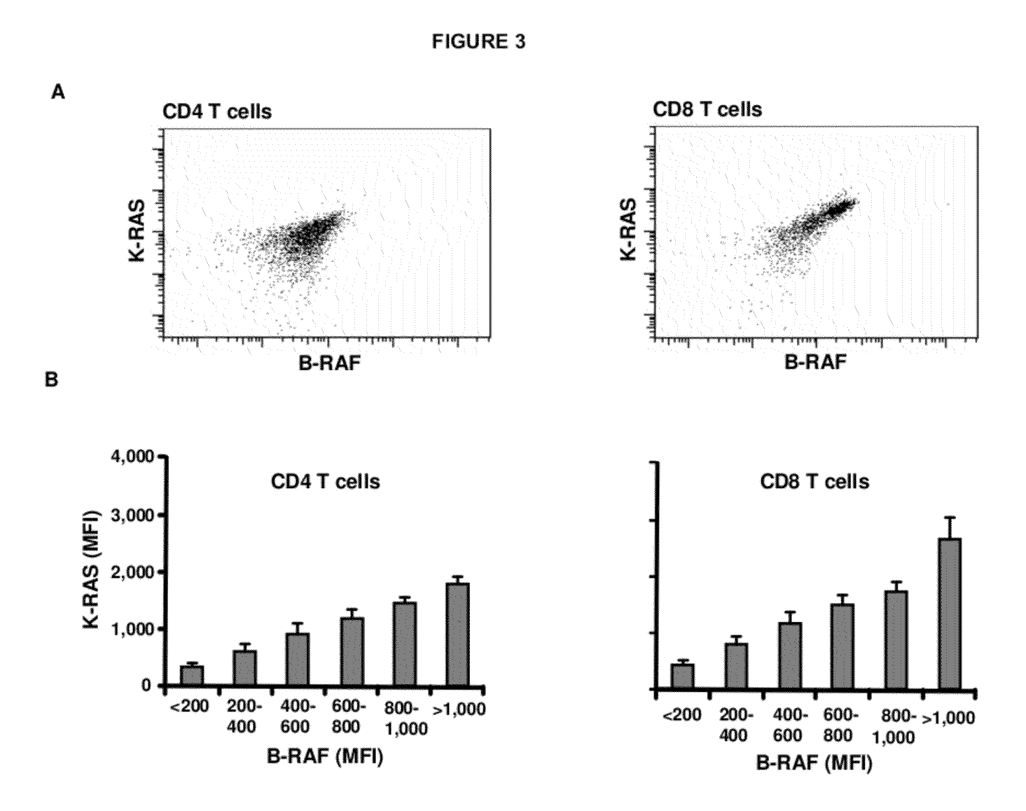

Correlation between B-Raf and K-Ras expression in single cells. Data so far were derived from total CD4 and CD8 T cell populations. Subset analysis showed that the increased ERK responsiveness in RA is not a feature of a small T cell subpopulation but is equally seen in naïve, memory and effector T cells. Our transcriptional studies identified at least two candidate molecules that may be responsible for the increased ERK activation in RA T cells. To determine whether the upregulation of K-Ras and B-Raf transcription is related, we compared K-Ras and B-Raf expression in CD4 and CD8 T cells at the single cell level. Representative scatter plots of K-Ras and B-Raf staining are shown in FIG. 3A. To quantify the correlation, T cells were divided into different windows based on their B-Raf expression, and K-Ras expression was determined for each window. B-Raf and K-Ras levels correlated in CD4 as well as CD8 T cells from RA patients (both p<0.001 by GEE regression analysis) suggesting shared transcriptional control mechanisms for both genes (FIG. 3B).

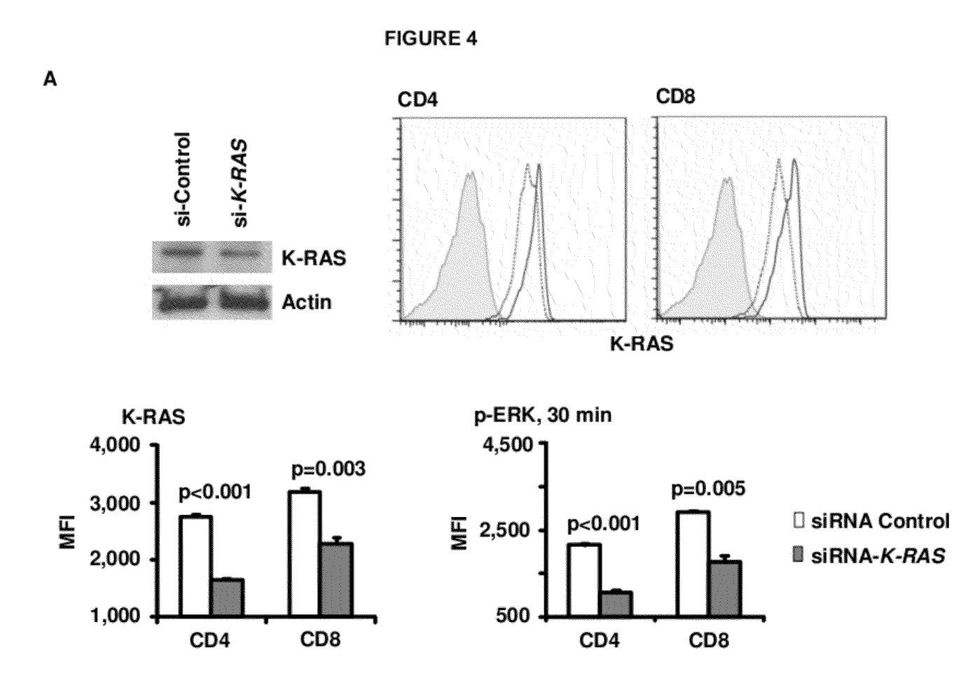

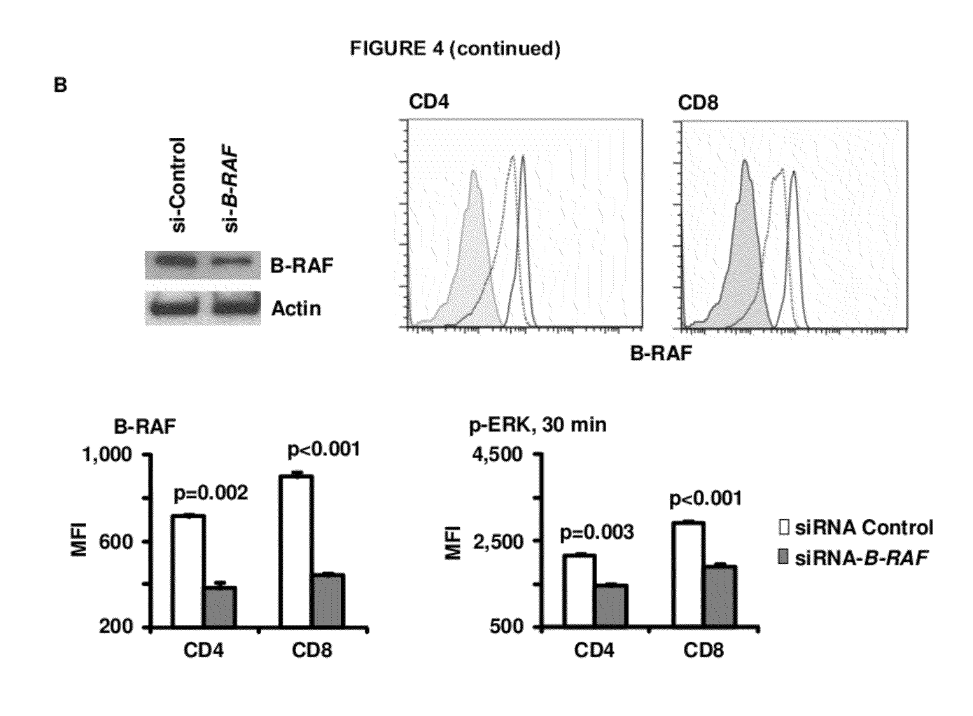

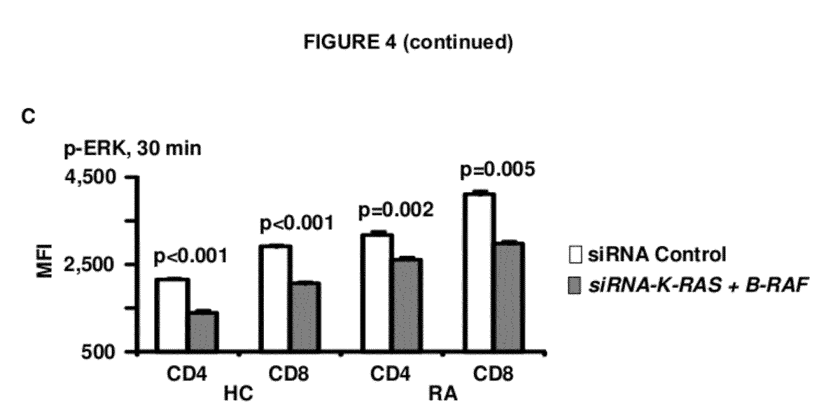

K-Ras and B-Raf concentrations in RA T cells influence ERK activation. The formation of several Ras/Raf complexes initiate the activation of the ERK pathway involving A-Raf, B-Raf, C-Raf, H-Ras, K-Ras and N-Ras depending on cell type and subcellular context. In T cells, C-Raf is thought to be dominant while B-Raf was thought to be completely absent and irrelevant or at least only relevant for selected functions in thymic selection. To determine whether B-Raf and K-Ras are critical for ERK phosphorylation in T cells, we silenced either signaling molecule or both molecules together. FIGS. 4A and B document a 50 to 70% reduction in expression both in CD4 and CD8 T cells 72 hours after transfection with specific siRNA compared to control siRNA as shown by Western blots as well as flow cytometry. The reduced K-Ras and B-Raf expression dampened the basal as well as activation-induced ERK phosphorylation in RA patients, clearly documenting that K-Ras and B-Raf levels directly regulate the basal and activation induced p-ERK levels in RA T cells. The partial silencing reduced ERK phosphorylation in RA T cells to the same level as in non-silenced control T cells (FIG. 4C).

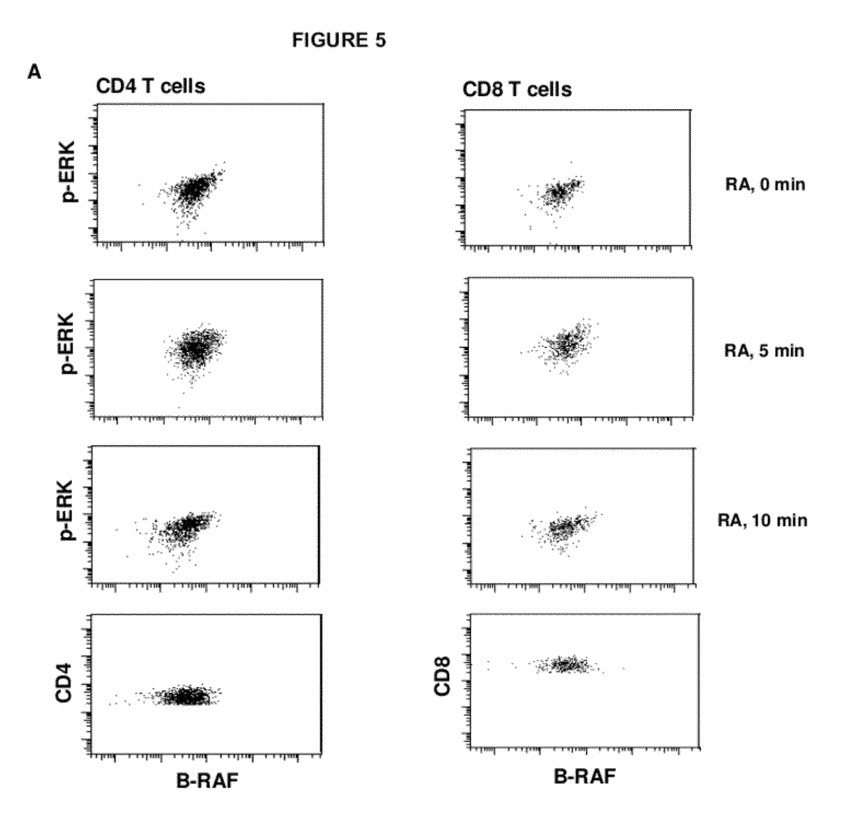

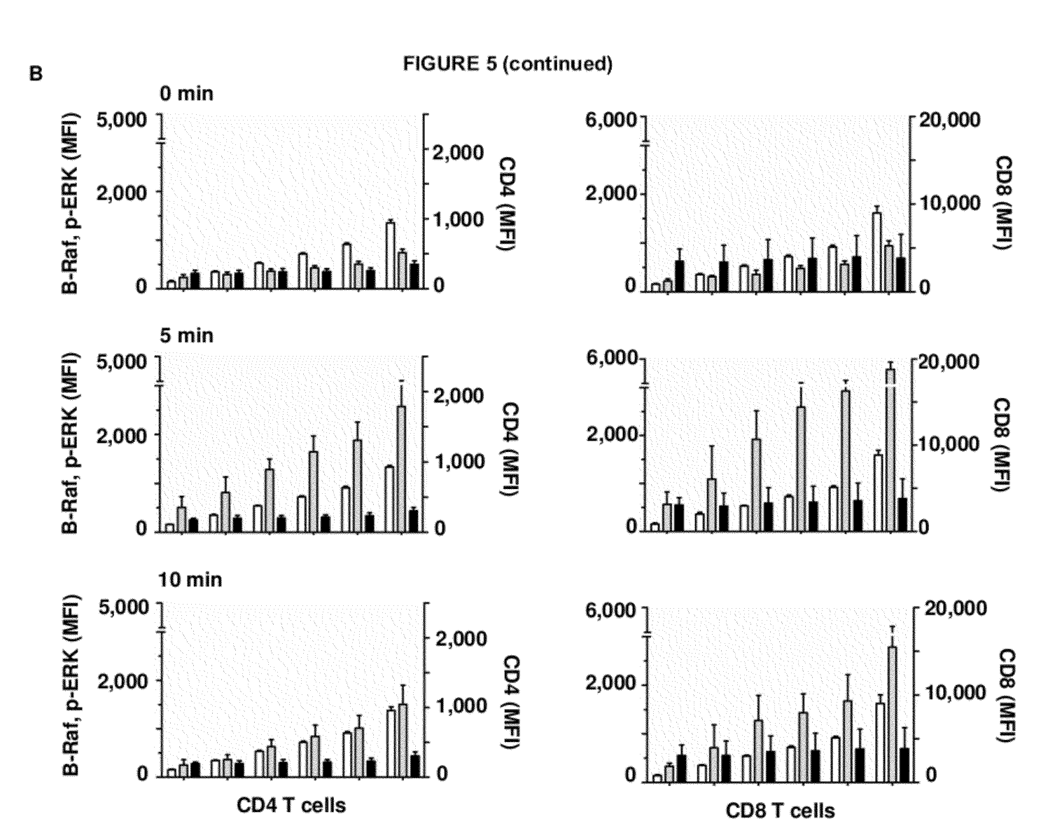

Silencing experiments showed that major reduction in B-Raf and K-Ras concentrations reduce but do not fully abrogate ERK phosphorylation. To address the question whether the smaller differences in expression observed between RA patients and healthy controls are of functional relevance, we used flow cytometry to correlate protein levels of signaling molecules and phosphorylation events. Based on B-Raf MFI, the T cell population was divided into six equal segments, and the expression of pERK in each gate was determined. CD4 and CD8 staining, respectively, was taken as internal control. As shown in FIG. 5, CD4 and CD8 T cells with higher B-Raf expression had increased basal levels of pERK and a more pronounced ERK response upon stimulation, clearly demonstrating that the B-Raf concentrations in T cells are critical and small differences have functional consequences. This correlation was specific as we did not see any correlation between B-Raf and CD4 levels in CD4 T cells or B-Raf and CD8 levels in CD8 T cells (FIG. 5).

Ras-Raf complex formation in RA patients. To monitor Raf activation, we examined Ras-Raf complex formation at the cell membrane of T cells stimulated with anti-CD3/CD28 antibodies. T cells were fixed at indicated times after TCR stimulation and stained with N-Ras or K-Ras (red) and B-Raf (green) specific antibodies.

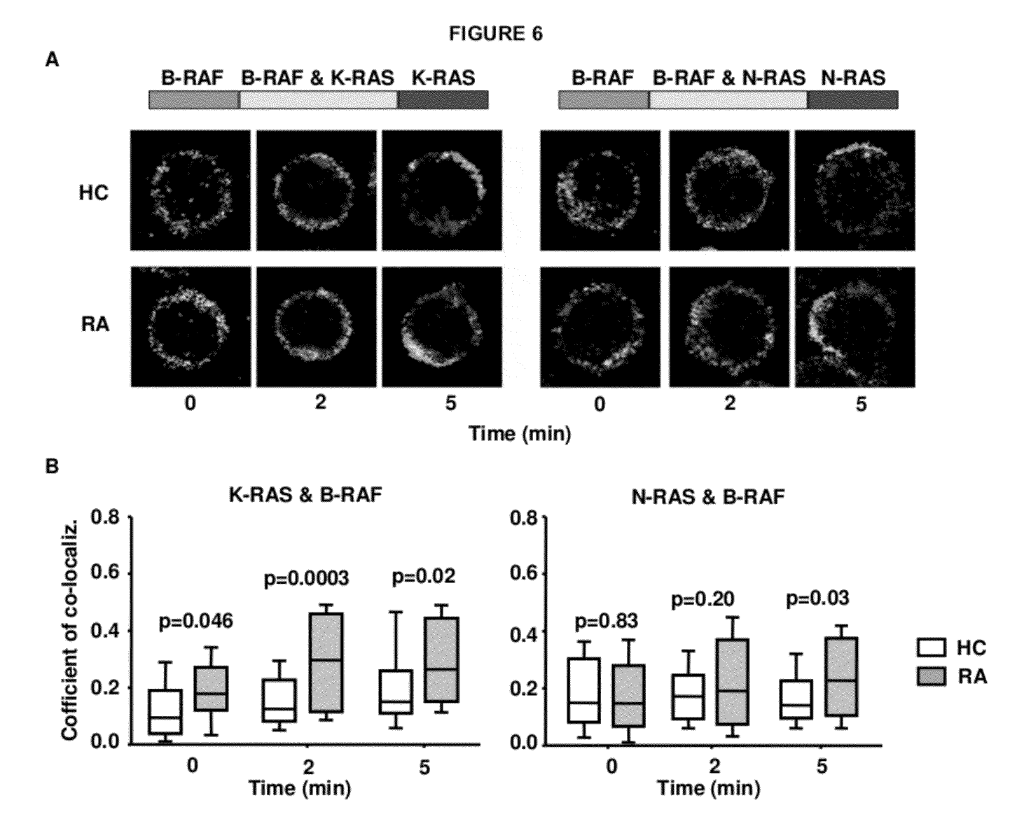

Membrane-close fluorescence was quantified in T cells, representative images are shown in FIG. 6A. Coefficients of colocalization were determined by regression analysis of the red and green pixel fluorescence and are summarized for five RA patients and five normal controls in FIG. 6B. In RA T cells, the coefficient for B-Raf/K-Ras was already slightly increased before stimulation compared to healthy controls, and exhibited a more pronounced response that was sustained over time. In contrast, colocalization of N-Ras and B-Raf was less induced by CD3 stimulation. A difference between control individuals and RA patients was seen at 5 minutes but not at earlier time points. The pattern of K-Ras or N-Ras complexing with C-Raf was identical and followed a similar kinetics as N-Ras/B-Raf; again a difference was seen for complex formation after 5 minutes. These data suggest a model of initially increased K-Ras/B-Raf activation with subsequent increased amplification loops involving N-Ras, K-Ras, and C-Raf.

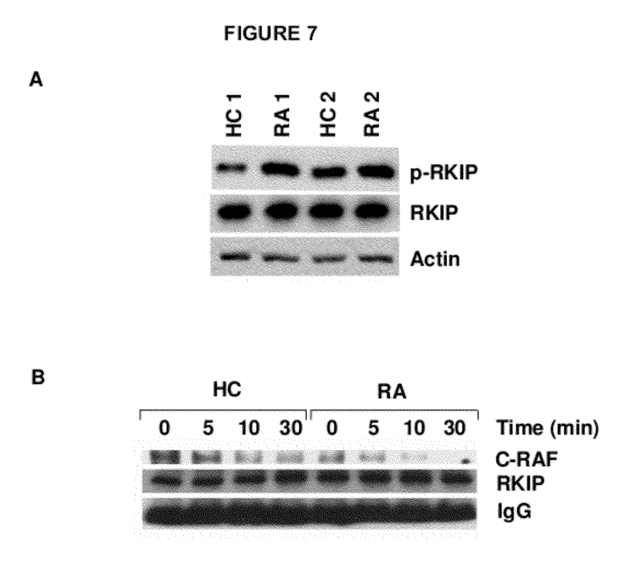

Activation of pERK amplification loops in RA T cells. The confocal studies suggested that the difference between RA patients and healthy controls can be mapped to increased B-Raf and subsequently sustained C-Raf activation in the patient. This model was supported by Western blotting for serine phosphorylated C-Raf. C-Raf phosphorylation was indistinguishable at 5 minutes, but higher in RA patients at subsequent time points. Bioavailability of Raf is controlled by several binding proteins, such as 14-3-3 and RKIP (22, 23). RKIP is involved in an important ERK amplification loop; RKIP binds to C-Raf preventing its activation. If phosphorylated, RKIP dissociates from C-Raf, which initiates a positive feedback loop of the ERK pathway. RKIP is phosphorylated by PKC or pERK. Increased pERK levels, therefore, can free-up more C-Raf in a positive feedback loop (24). In contrast, RKIP does not sequester B-Raf. Western blots showed that RA patients have higher basal p-RKIP levels compared to control individuals while having similar total RKIP levels (FIG. 7A). RKIP was immunoprecipitated and the precipitates were examined for C-Raf by Western blotting. The amount of RKIP-bound C-Raf was higher in normal individuals than in RA patients before and after stimulation (FIG. 7B), suggesting that the activation of this positive feedback loop is increased in RA patients.

It is shown herein that T cells from RA patients have increased cytoplasmic concentrations of the signaling molecules B-Raf and K-Ras, causing hyperreactivity of the ERK pathway upon TCR stimulation. ERK activity has been shown to be an important regulator of TCR threshold calibration. B-Raf is of interest because its expression levels in T cells are normally minute and increased expression alleviates the need for costimulation and impairs anergy induction. The increased expression of B-Raf and K-Ras accounts for immunological findings including the autoreactivity as well as accelerated T cell aging characteristic of RA.

C-Raf has been considered as the major Raf kinase in T cells, while B-Raf is best known as an oncogene in melanoma and other tumors. In contrast to A-Raf and C-Raf, B-Raf is constitutively phosphorylated at position S445 and only requires membrane recruitment but no phosphorylation for activation, which may explain its unique role as an oncogene. In T cells, B-Raf was originally considered to be completely absent, but has recently been shown to be functional in thymic selection. In chimeric mice reconstituted with B-Raf deficient stem cells, T cell development is drastically stopped at the stage of double-positive thymocytes and the maturation into CD4 and CD8 single positive cells is highly impaired, suggestion a role of B-Raf mediated ERK activation in positive selection. In mature T cells, ectopic B-Raf expression prevents anergy induction. T cells transfected with B-Raf and stimulated under anergizing conditions activated the ERK pathway, produced IL-2 and proliferated. The ability of B-Raf to be anti-anergic may relate to its intrinsic activity. B-Raf recruited to active RAP1 activates the ERK pathway without the need for additional phosphorylation, whereas C-Raf bound to RAP-1 is inactive and therefore functionally silenced.

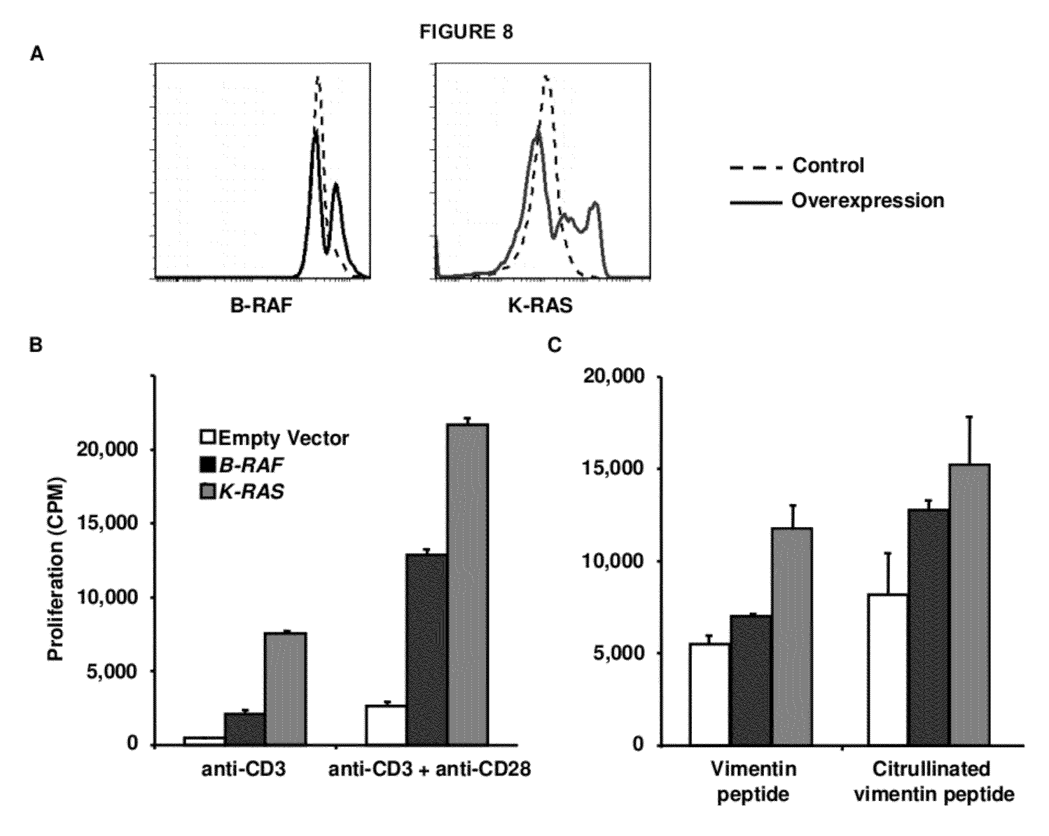

The data provided herein suggests that that increased B-Raf expression lowers TCR activation threshold in T cells from RA patients and renders these cells more susceptible to activation. Overexpression of K-Ras may play a complementary role. Immediately after activation, increased K-Ras expression was not sufficient to increase membrane recruitment of C-Raf, however, increased co-clustering of K-Ras and C-Raf was seen after five minutes, possibly due to a positive feedback loop with ERK-dependent RKIP phosphorylation and release of bioavailable C-Raf. Our functional assays showed that forced overexpression of B-Raf or K-Ras alone heightens a T cell response to CD3 and CD28 cross-linking (FIG. 8A) and lowers the TCR threshold sufficiently to permit the activation of self-reactive T cells to vimentin and, even more, to citrullinated vimentin (FIG. 8B). Increased TCR sensitivity to stimulation with low affinity antigens and relative resistance to anergy induction explain many of the T cell phenomena observed in RA. Increased TCR sensitivity could increase homeostatic proliferation, facilitate differentiation into CD28⁻ effector cells and accelerate cell aging and repertoire. At the same time, it may facilitate responses to antigens that are controlled by peripheral tolerance mechanisms and not by central negative selection, such as responses to neoantigens.

B-Raf is as an excellent target to prevent or treat rheumatoid arthritis because its inhibition can be selectively be directed at a signaling abnormality in RA T cells while leaving C-Raf mediated ERK activation intact.

What is claimed is:

1. A method for the prognosis or diagnosis of a patient for the development of an autoimmune disease, the method comprising:
determining the expression of one or both of B-Raf and K-Raf in a cell population comprising T cells obtained from said patient;
comparing the expression with the expression of a control cell population; wherein increased expression relative to a normal cell is indicative that said patient is at risk for disease development.

2. The method according to claim 1, wherein said patient has shown symptoms of autoimmune disease.

3. The method of claim 1, wherein the autoimmune disease is rheumatoid arthritis.

4. The method according to claim 1, wherein said cell population is obtained from blood.

5. The method of claim 1, wherein said cell population is enriched for T cells.

6. The method of claim 1, wherein the T cells are activated.

7. The method according to claim 1, wherein said diagnosis or prognosis is utilized to select a specific therapeutic agent to treat said patient.

8. The method according to claim 1, wherein said patient is a human.

9. A method of treating an autoimmune disease, the method comprising:
administering to a patient suffering from the autoimmune disease a selective inhibitor of B-Raf and/or K-ras in a dose sufficient to inhibit defects in T cell signaling pathways associated with autoimmune disease.

10. The method of claim 9, wherein the autoimmune disease is rheumatoid arthritis.
